(19) United States
(12) Patent Application Publication
(10) Pub. No.: US 2014/0106421 A1
(43) Pub. Date: Apr. 17, 2014

(54) Methods Of Producing Carboxylic Acids And/Or Alcohols

(71) Applicant: Industrial Technology Research Institute, Hsinchu (TW)

(72) Inventors: Hsiu-Yin YIN, Zhubei City (TW); Yun-Huin LIN, Hsinchu City (TW); Chih-Fen Lu, New Taipei City (TW); Ying-Ching Su, Guishan Township (TW); Jheng-Jin Luo, Zhudong Township (TW); Hom-Ti Lee, Zhubei City (TW)

(21) Appl. No.: 14/054,520

(22) Filed: Oct. 15, 2013

Related U.S. Application Data
(60) Provisional application No. 61/713,840, filed on Oct. 15, 2012.

(51) Int. Cl. C12P 7/52

(52) U.S. Cl. CPC C12P 7/52
USPC 435/140; 435/136; 435/157; 435/141; 435/145; 435/144; 435/161; 435/160

(57) ABSTRACT

Carboxylic acids and/or alcohols are produced by a fermentation process comprising: growing an immobilized microorganism capable of producing carboxylic acids and/or alcohols in an aqueous medium and in the presence of an organic medium, and recovering the carboxylic acids and/or alcohols from the organic medium; wherein a mesh is placed at an interface of the organic medium and the aqueous medium; and further wherein the organic medium comprises at least one organic solvent and at least one extractant chosen from tri-alkylphosphine oxides and tri-alkylamines.

This application claims priority to U.S. Provisional Application No. 61/713,840, filed on Oct. 15, 2012.

Carboxylic acids/alcohols can be produced via either chemical synthetic methods in which petroleum oil or gas may be used as a starting material or biological methods such as fermentation of sugar or starch.

Biological methods have certain advantages as compared to chemical synthetic methods. For one, the petroleum material commonly used in the chemical synthetic methods is a nonrenewable resource and may lead to the production of unwanted byproducts.

During the fermentation process that may be used in biological methods, a microorganism produces and releases carboxylic acids/alcohols into the fermentation medium. After reaching certain concentrations in the fermentation medium, however, carboxylic acids/alcohols produced may inhibit the activity and productivity of the microorganism, thereby limiting the overall yield of the fermentation process. Such phenomenon is often referred to as the end-product inhibition.

One approach to mitigate the end-product inhibition is to separate out carboxylic acids and/or alcohols from the fermentation broth during the fermentation so that they would not reach an inhibitory concentration. However, some of the separation processes commonly used for removing carboxylic acids/alcohol (e.g., distillation or precipitation process) may lead to a higher manufacturing cost, generate unwanted solid wastes, and/or reduce the overall efficiency of the process.

In some embodiments herein, a fermentation process is disclosed for producing carboxylic acids and/or alcohols with an integrated solvent extraction process that may alleviate the concern of the end-product inhibition. In some embodiments, the disclosed process may be more efficient and cost-effective than traditional fermentation processes that rely primarily on distillation and/or a precipitation process for recovering carboxylic acids/alcohols.

Additional features and advantages of the present disclosure will be set forth in part in the description which follows, and in part will be obvious from the description, or may be learned by practice of the invention. The features and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

The accompanying drawings, which are incorporated in and constitute a part of this specification, illustrate embodiments of the disclosure and together with the description, serve to explain the principles of the certain embodiments.

### BRIEF DESCRIPTION OF THE DRAWINGS

FIGS. 1a and 1b provide schematic diagrams of exemplary fermentation processes conducted in a continuous mode.

FIG. 2 provides a schematic diagram of an extractive fermentation-column reactor.

FIG. 3 shows concentrations of glucose, butyric acid, propionic acid, and OD₆₀₀ in the aqueous medium of the fermentation process described in Example 4A.

As described in Example 4F, FIGS. 4A-C show the concentrations of glucose, acetic acid, butyric acid, and propionic acid in the aqueous medium measured at different time points, as well as the concentration of butyric acid in the organic medium after about 115 hours of fermentation; the immobilized microorganism were PVA-immobilized C. tyrobutyricum ITR104001 (FIG. 4A), C. butyricum ITR104003 (FIG. 4B), or C. tyrobutyricum ITR104004 (FIG. 4C).

FIG. 5 provides a schematic diagram of the extractive fermentation-column reactor described in Example 5.

FIG. 6 shows the concentrations of glucose, butyric acid, and propionic in the basal medium (aqueous medium) at various time points and in a reactor that contained only oleyl alcohol as the organic phase, as described in Example 6.

FIG. 7 shows the concentrations of glucose, butyric acid, and propionic in the basal medium (aqueous medium) at various time points and in the reactor that contained 1 M TOPO in oleyl alcohol as the organic phase, as described in Example 6.

### DESCRIPTION OF THE EMBODIMENTS

Reference will now be made in detail to the present embodiments examples of which are illustrated in the accompanying drawings.

The present disclosure provides a fermentation process for making carboxylic acids and/or alcohols that may alleviate concerns with end-product inhibition.

In some embodiments, the fermentation process for making carboxylic acids and/or alcohols comprises: growing an immobilized microorganism capable of producing carboxylic acids and/or alcohols in an aqueous medium and in the presence of an organic medium, and recovering the carboxylic acids and/or alcohols from the organic medium; wherein a solid mesh is placed at the interface of the organic medium and the aqueous medium; and further wherein the organic medium comprises at least one organic solvent and at least one extractant chosen from tri-alkylphosphine oxides and tri-alkylamines.

In some embodiments, the fermentation process disclosed herein may be used to produce carboxylic acids such as acetic, propionic, butyric, fumaric, malic, acrylic, citric, gluconic, itaconic acid, or mixtures thereof. For example, the fermentation process disclosed herein may be used to produce butyric acid.

In some embodiments, the fermentation process disclosed herein may be used to produce alcohols such as ethanol, propanol, butanol, or mixtures thereof. For example, the fermentation process disclosed herein may be used to produce butanol.

In some embodiments, the microorganism capable of producing carboxylic acids and/or alcohols is chosen from bacterias capable of producing carboxylic acids such as butyric acid. In some embodiments, the microorganism is chosen from C. tyrobutyricum, C. thermobutyricum, C. butyricum, C. populeti, C. cadaveros, C. cellobioparum, C. cochlearium, C. pasteurianum, C. roseum, C. rubrum, and C. sporogenes.

In some embodiments, the microorganism capable of producing carboxylic acids and/or alcohols is chosen from bacterias capable of producing alcohols such as butanol. In some embodiments, the microorganism is chosen from C. acetobutyricum, C. beijerinckii, Clostridium aurantibutyricum, and C. tetanomorphum.

In some embodiments, the microorganism capable of producing carboxylic acids and/or alcohols is chosen from bacterias capable of converting lactic acid into butyric acid.

In some embodiments, the microorganism capable of producing carboxylic acids and/or alcohols may be cultivated with any culture media, substrates, conditions, and processes generally known in the art for culturing bacteria.

Depending on the specific requirements of the microorganism, attention should be given to select appropriate growth medium, pH, temperature, agitation rate, inoculum level, and/or aerobic, microaerobic, or anaerobic conditions for the fermentation process.

For example, in some embodiments, the microorganism may need to be cultured under anaerobic conditions. “Anaerobic conditions” as used herein means the level of oxygen (O₂) is below 0.5 parts per million in the gas phase. In that case, the aqueous medium used for the fermentation process may need to be conditioned or purged with an oxygen-free gas such as nitrogen gas to reduce the level of oxygen in the media.

Further as a non-limiting example, the microorganism may be cultured at a temperature ranging from about 20° C. to about 80° C. For instance, the microorganism may be cultured at 37° C.

In the fermentation process, the microorganism is inoculated into the aqueous medium in an immobilized form. In some embodiments, immobilized microorganism is prepared by entrapping microorganism in a natural or synthetic polymer. Natural or synthetic polymers suitable for entrapping microorganism include but not limited to cellulose acetate, polystyrene, polyvinyl alcohol, or polyurethane. In some embodiments, methods and materials described in “Immobilization of Enzymes and Cells,” Methods in Biotechnology, edited by G. F. Bickerstaff, vol. 1 Humana Press, Totowa N.J. (1997), may be used to prepare the immobilized microorganism.

In some embodiments, the immobilized microorganism is prepared by encapsulating microorganism in a hydrogel such as agar, gelatin, or alginate.

In some embodiments, the immobilized microorganism is prepared by immobilizing microorganism in phosphorylated polyvinyl alcohol (PVA) gel beads according to a method described in Chen, K. C., “Immobilization of microorganism with phosphorylated polyvinyl alcohol (PVA) gel,” Enzyme Microbio. Technol. 1994, 16, 679-83.

In some embodiments, for the growth of the microorganism and for its production of carboxylic acids/alcohols, the aqueous medium may comprise any nutrients, ingredients, and/or supplements suitable for growing the microorganism or for promoting the production of the desired products. As a non-limiting example, the aqueous medium may comprise at least one ingredient chosen from: carbon sources, including but not limited to monosaccharides (such as glucose, galactose, fructose, xylose, arabinose, or xylulose), disaccharides (such as lactose or sucrose), oligosaccharides, and polysaccharides (such as starch or cellulose), one-carbon substrates and/or mixtures thereof; nitrogen sources, such as an ammonium salt, yeast extract, or peptone; minerals; salts; cofactors; buffering agents; vitamins; and any other components and/or extracts that may promote the growth of the bacteria. Further as a non-limiting example, for the microorganism to produce carboxylic acids such as butyric acid, materials and methods described in Dwidar M. et al., “The Future of Butyric Acid in Industry,” The Scientific World Journal, Vol. 2012, Article ID 471417 (2012), may be used.

In some embodiments, the pH of the aqueous medium may range from about 3.0 to about 9.0. As a non-limiting example, depending on the ionization constant of the desired products and the growth of the microorganism, the pH of the aqueous medium may range from about 3.0 to about 6.0. In some embodiments, the pH of the aqueous medium is maintained in a range of about 3.0 to about 6.0 by adding acids/basics during the fermentation.

In the fermentation process, the microorganism is grown in the aqueous medium and in the presence of an organic medium separated from the aqueous medium. The organic medium is capable of extracting out the carboxylic acids and/or alcohols from the aqueous medium during the fermentation.

In some embodiments, the organic medium comprises at least one organic solvent and at least one extractant chosen from tri-alkylphosphine oxides and tri-alkylamines.

In some embodiments, the at least one organic solvent is chosen from C₈-C₁₈ alcohols, triglyceride, C₆-C₁₆ alkanes, and fatty acid methyl esters.

In some embodiments, the at least one organic solvent is oleyl alcohol.

In some embodiments, the at least one organic solvent is chosen from hexane and soybean oil.

In some embodiments, the at least one extractant is chosen from trioctylamine (TOA) and trioctylphosphine oxide (TOPO).

In some embodiments, the concentration of the at least one extractant in the organic medium is less than or equal to 1 M, such as less than or equal to 0.9 M, 0.8 M, 0.7 M, 0.6 M, or 0.5 M.

In some embodiments, the organic medium comprises trioctylamine and oleyl alcohol.

In some embodiments, the organic medium comprises trioctylphosphine oxide and oleyl alcohol.

The volume ratio of the organic medium and the aqueous medium in the reactor may depend on a number of factors, including but not limited to, the type of microorganism grown, the size of the reactor, the partition coefficient of the solvent for the product, and the fermentation mode chosen, as described below.

In some embodiments, the volume ratio of the organic medium to the aqueous medium may range from 1:5 to 20:1. In some embodiments, the organic medium to the aqueous medium may range from 5:1 to 15:1.

In the fermentation process, a solid mesh is placed in the reactor to avoid the immobilized microorganism from getting into direct contact with the organic medium.

In some embodiments, a solid mesh is placed at the interface of the organic medium and the aqueous medium.

In some embodiments, the solid mesh is fabricated from metals or polymers. In some embodiments, to prevent microorganism from contacting with extractant, the pore size of the mesh may be less than the diameter of the immobilized cell beads.

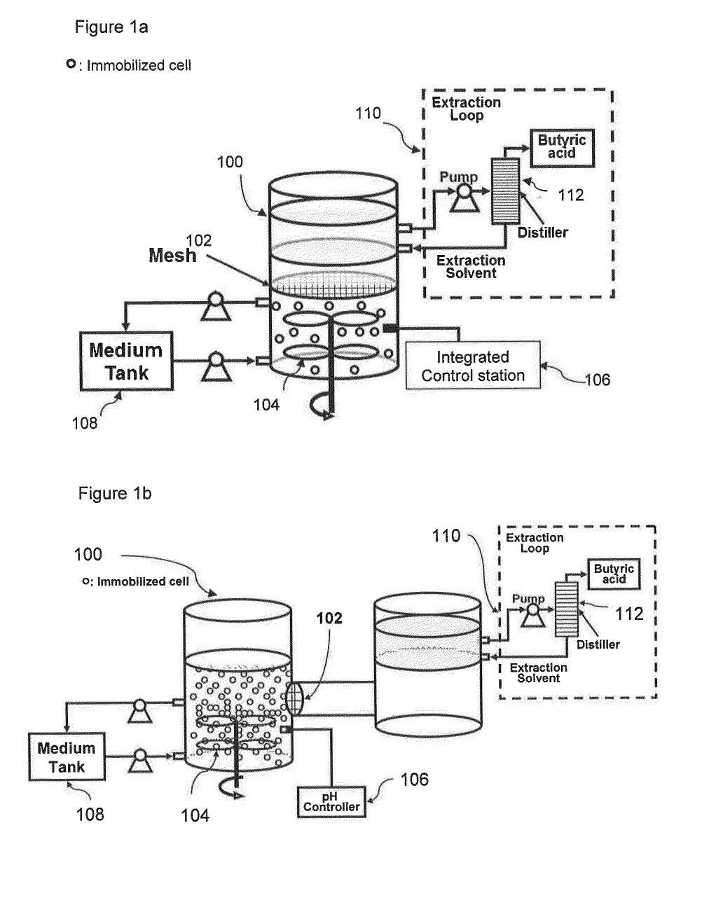

In some embodiment, the fermentation process may be carried out in a continuous mode with a stirred-tank fermentor, such as a stirred-tank reactor 100 generally illustrated in FIG. 1a. In some embodiments, the reactor can be set up as a sequential reactor, illustrated in FIG. 1b.

As a non-limiting example, the stirred-tank reactor 100 used for growing the immobilized microorganism may be equipped with a round mesh 102 made of stainless steel, a mechanical stirrer 104, a thermometer (not shown), and a pH meter (not shown). The stirrer, thermometer, and pH meter may all be connected to and controlled by an integrated control station 106. The round mesh 102 is placed in between the aqueous medium and the organic medium to avoid the immobilized microorganism from contacting directly with the organic medium.

Further as a non-limiting example, a peristaltic pump may be used to continuously pump the aqueous medium in between a medium tank 108 and the reactor 100. For example, the aqueous medium may be continuously introduced from the medium tank 108 into the reactor 100 and from the reactor 100 back to the medium tank 108. Alternatively, the aqueous medium may be continuously removed from the reactor, and fresh aqueous medium continuously added into the reactor 100.

Also further as a non-limiting example, the organic medium containing the carboxylic acids/alcohols may be pumped via an extraction loop 110 to a separation apparatus such as a distillation column 112 to recover the carboxylic acids/alcohols from the organic medium. After separation, the at least one organic solvent and/or extractant may then be recycled back into the fermentor for further extraction of the carboxylic acids/alcohols. Alternatively, fresh organic medium may be continuously added to the fermentor to replenish the removed organic medium.

In the continuous mode of the fermentation process, because the product is continually removed from the reactor, a smaller volume of organic medium may be required, thus enabling a larger volume of the fermentation broth (aqueous medium) to be used. This may lead to a higher product yields.

In some embodiments, the fermentation process may be carried out in a batchwise fermentation mode. In this mode, neither the aqueous medium nor the organic medium is removed from the reactor during the fermentation process. While this mode is simpler than the continuous mode described above, it may require a larger volume of organic medium to minimize the concentration of the inhibitory product in the aqueous medium. Consequently, the volume of the aqueous medium may need to be smaller than the volume of the organic medium in the reactor.

Recovery of the carboxylic acid/alcohols from the organic medium may be done by any methods known in the art, including but not limited to, distillation, adsorption by resins, separation by molecular sieves, or precipitation. In some embodiments, the carboxylic acids/alcohols is recovered from the organic medium by distillation.

Other embodiments will be apparent to those skilled in the art from consideration of the specification and practice of the embodiments disclosed herein. It is intended that the specification and examples be considered as exemplary only, with a true scope and spirit of the invention being indicated by the following claims.

### EXAMPLES

### Example 1

### Extraction of Butryic Acid from CGM Fermentation Media

### Example 1.1

50 ml of CGM culture medium (basal medium, see the table provided immediately below) containing 5% of butyric acid (the pH of the CGM medium decreased to 4 after the addition of the butyric acid) was mixed with 5 ml of 0.7 M, 1 M, 011.4 M of trioctylamine (C₂₄H₅₁N, CAS No. 1116-76-3, abbreviation: TOA) in oleyl alcohol at 37° C.

Clostridium Growth Medium (CGM)

| | |
|---|---|
| Yeast extract | 5 g/L |
| Peptone | 5 g/L |
| (NH₄)₂SO₄ | 3 g/L |
| K₂HPO₄ | 1.5 g/L |
| MgSO₄•7H₂O | 0.6 g/L |
| FeSO₄•7H₂O | 0.03 g/L |

Final pH: 6.0 ± 0.2 at 25° C.

The concentrations of butyric acid in the CGM culture medium (i.e., the aqueous phase) and in the trioctylamine solution (i.e., the organic phase) were measured every 12 hours by high performance liquid chromatography (HPLC).

Table 1 shows the butyric acid distribution ratios from the CGM medium extracted with different concentrations of trioctylamine.

TABLE 1

| | Concentration of TOA | | |
|---|---|---|---|
| | 0.7M | 1M | 1.4M |
| Butyric acid distribution ratio (D) | 7.38 | 10.21 | 13.82 |

The butyric acid distribution ratio (D) was calculated by dividing the concentration of butyric acid in the organic medium by the concentration of butyric acid in the aqueous medium:

$$D = \frac{[ butyric acid ] organic medium}{[ butyric acid ] aqueous medium}$$

When comparing the extraction efficiency of different organic solvents, the higher the distribution ratio, the better the extraction efficiency.

Table 1 shows that the extraction efficiency increases with the concentration of trioctylamine in the organic phase.

### Example 1.2

10 ml of CGM culture medium containing 1%, 2%, or 5% of butyric acid was mixed with 10 ml of 0.1 M, 0.5 M, or 1 M of trioctylphosphine oxide (C₂₄H₅₁OP, CAS No. 78-50-2, abbreviation: TOPO) in oleyl alcohol (C₁₈H₃₆O) at 37° C. The pH of the culture medium was further adjusted to a value ranging from 4 to 6 with 4 N NaOH and 4 N HCl.

After 24 hours, the concentrations of butyric acid in the CGM culture medium (the aqueous phase) and in the TOPO solution (the organic phase) were measured by HPLC.

Table 2 shows the butyric acid distribution ratios from the CGM medium extracted with different concentrations of TORO.

TABLE 2

| | | pH | | |
|---|---|---|---|---|
| Butyric acid (%) | TOPO (M) | 4 | 5 | 6 |
| 1 | 0.1 | 1.45 | 0.31 | 0.00 |
| 2 | | 2.12 | 0.37 | 0.05 |
| 3 | | 2.17 | 0.26 | 0.00 |
| 1 | 0.5 | 1.91 | 0.36 | 0.03 |
| 2 | | 2.82 | 0.37 | 0.06 |
| 3 | | 2.90 | 0.35 | 0.04 |
| 1 | 1 | 2.67 | 0.42 | 0.06 |
| 2 | | 3.88 | 0.43 | 0.04 |
| 3 | | 4.20 | 0.35 | 0.04 |

### Example 1.3

10 ml of CGM culture medium containing 1%, 2%, or 5% of butyric acid was mixed with 10 ml of 0.1 M, 0.5 M, or 1 M of tributylphosphate (C₁₂H₂₇O₄P, CAS No, 126-73-8, abbreviation: TBP) in oleyl alcohol at 37° C. The pH of the culture medium was further adjusted to a value ranging from 4 to 6 with 4 N NaOH and 4 N HCl.

After 24 hours, the concentrations of butyric acid in the CGM culture medium (the aqueous phase) and in the TBP solution (the organic phase) ere measured by HPLC.

Table 3 shows the butyric acid distribution ratios from the CGM medium extracted with different concentrations of TOPO.

TABLE 3

| | | pH | | |
|---|---|---|---|---|
| Butyric acid (%) | TBP (M) | 4 | 5 | 6 |
| 1 | 0.1 | 1.77 | 0.51 | 0.05 |
| 2 | | 2.34 | 0.51 | 0.05 |
| 3 | | 2.31 | 0.44 | 0.04 |
| 1 | 0.5 | 2.04 | 0.55 | 0.10 |
| 2 | | 2.75 | 0.56 | 0.05 |
| 3 | | 2.64 | 0.46 | 0.05 |
| 1 | 1 | 2.57 | 0.62 | 0.08 |
| 2 | | 3.47 | 0.61 | 0.07 |
| 3 | | 3.20 | 0.50 | 0.05 |

### Example 2

A culture medium was prepared by dissolving 12 mg of glucose, 4 mg of yeast extract, 4 mg of peptone, 2.4 mg of (NH₄)₂SO₄, 1.2 mg K₂HPO₄, 0.48 mg of MgSO₄.7H₂O, and 0.024 mg of FeSO₄.7H₂O in 0.8 ml of water. The culture medium was then inoculated with 0.08 ml of bacterial stock comprising C. tyrobutyricum ATCC25755. The initial optical density (OD) measured at a wavelength of 600 nm was 0.2. After inoculation, 0.8 ml of an organic solution (as listed in the Table 4 below) was added to the top of the culture medium. The bacteria was then cultured in Tecan infinite 200 Pro Multimode Microplate Readers (Tecan Group Ltd., Switzerland) for 24 hours at 37° C., with shaking once every 30 minutes.

As shown in Table 4 below, the growth rate and the glucose consumption rate of the bacteria were rather insignificant in the presence of organic medium containing TOPO and (1) oleyl alcohol, (2) isoamyl alcohol, or (3) cyclohexyl acetate as the solvent.

TABLE 4

| | | | | Concentration of the |
|---|---|---|---|---|
| | | Bacterial | Glucose | butryic acid in the |
| Components of the organic | Bacteria | Growth rate | consumption | aqueous phase |
| solution added to the culture | growth | (OD/h) | rate % | g/L |
| None | + | 0.05 | 70.7 | 7.2 |
| Oleyl alcohol only | + | 0.08 | 80.7 | 4.7 |
| 150 mg of TOPO dissolved in | + | — | 31.4 | 1.6 |
| 0.4 ml of Oleyl alcohol | | | | |
| 150 mg TOPO dissolved in 0.4 ml | + | 0.05 | 95.1 | 1.5 |
| of hexane | | | | |
| 150 mg of TOPO dissolved in | + | — | 10.4 | 0.5 |
| 0.4 ml of isoamyl alcohol | | | | |
| 150 mg of TOPO dissolved 0.4 ml | + | — | 10.6 | 1.2 |
| of cyclohexyl acetate | | | | |

### Example 3

A culture medium was prepared by dissolving 12 mg of glucose, 4 mg of yeast extract, 4 mg of peptone, 2.4 mg of (NH₄)₂SO₄, 1.2 mg K₂HPO₄, 0.48 mg of MgSO₄.7H₂O, and 0.024 mg of FeSO₄.7H₂O in 0.8 ml of water. The culture medium was then inoculated with 0.08 ml of bacterial stock comprising C. tyrobutyricum ATCC25755. The initial optical density (OD) measured at a wavelength of 600 nm was 0.2. After inoculation, 0.8 ml of an organic solution (listed in the Table 4 below) was added to the top of the culture medium. The bacteria was then cultured in Tecan infinite 200 Pro Multimode microplate readers (Tecan Group Ltd., Switzerland) for 24 hours at 37° C., with shaking once every 30 minutes.

As shown in Table 5 below, when TOA/hexane organic solution was added to the culture, the bacteria was not able to grow or produce butyric acid.

In addition, when oleyl alcohol or soybean oil was used as a solvent for the organic solution, no detectable cell growth or glucose consumption rate was observed with 1 M of TOA. Moreover, it appears that soybean oil could hardly extracted out any butyric acid into the organic phase.

TABLE 5

| | | | | | Concentration | Concentration | |
|---|---|---|---|---|---|---|---|
| | | | Bacterial | | of the butryic | of the butryic | |
| | | | Growth | Glucose | acid in the | acid in the | Butyric acid |
| Components of the | | Bacteria | rate | consumption | aqueous phase | organic phase | distribution |
| Organic solution | | growth | (OD/h) | rate % | g/L | g/L | ratio |
| 0.5M | Oleyl alcohol | + | 0.18 | 100 | 1.2 | 1.4 | 1.12 |
| TOA | Soybean oil | + | 0.11 | 21.5 | 0.6 | 0 | — |
| | hexane | − | 0 | — | — | — | — |
| 0.7M | Oleyl alcohol | + | 0.04 | 100 | 1.2 | 1.7 | 1.38 |
| TOA | Soybean oil | + | 0.01 | 21.2 | 0.72 | 0 | — |
| | hexane | − | 0 | — | — | — | — |
| 1M | Oleyl alcohol | − | 0 | — | — | — | — |
| TOA | Soybean oil | − | 0 | — | — | — | — |
| | hexane | − | 0 | — | — | — | — |

### Example 4

### Bacteria Culture

Clostridium tyrobutyricum (ATCC 25755) purchased from Bioresource Collection and Research Center (BCRC) was used in this example. The stock culture was initially kept in serum bottles under anaerobic conditions at 4° C. and later pre-cultured in a serum bottle containing 100 ml of Reinforced Clostridial Medium (RCM, Merck) anaerobically with agitation at 37° C. for 48 hours prior to use.

The basal medium contained the following ingredients per liter of deionized water: 5 g of yeast extract, 5 g of peptone, 3 g of ammonium sulphate, 1.5 g of KH₂PO₄, 0.6 g of MgSO₄.7H₂O, and 0.03 g of FeSO₄.7H₂O. (Wu et al., Biotechnology and Bioengineering 2003, 82(1), 93-102.) The feedstock was prepared by adding appropriate substrates to the basal medium. All media and feedstocks were sterilized by autoclaving at 121° C., 15 psig, for 30 minutes prior to use.

### Cell Immobilization

To grow the cells for immobilization, a 5-L fermentor filled with 4 liter of the basal medium containing glucose and lactic acid as the substrates was inoculated with about 300 ml of the cell suspension prepared with the serum bottles described above. The cells were then allowed to grow for 7 days until the cell concentration reached an optical density (OD₆₀₀) of about 5.

The cells were immobilized in phosphorylated polyvinyl alcohol (PVA) gel beads according to the method described in Chen, K., “Immobilization of microorganism with phosphorylated polyvinyl alcohol (PVA) gel,” Enzyme Microbio. Technol. 1994, 16, 679-83. The cells were harvested by centrifugation at 6,500 rpm (KUBOTA, model 7780) for 10 min and suspended in 9% (w/v) PVA aqueous solution at a ratio of 20 g wet cell per liter of PVA solution. After thoroughly mixing the cell suspension and the PVA solution, the PVA-cell mixture was added to a saturated boric acid and sodium phosphate solution and gently stirred for 1-2 hours to form spherical beads. The resulting beads with a diameter of 3 mm to 4 mm were rinsed with water.

### Analysis

Free cell density was analyzed by measuring the optical density of the cell suspension at a wavelength of 600 nm (OD₆₀₀) with a spectrophotometer (OPTIZEN, model 2120UV plus).

Analysis of the liquid products, for example, organic acids and carbohydrates, was performed on HPLC (Agilent HP-1100) equipped with Aminex HPX-87H column (300×7.8 mm), a column oven at 75° C., and a refractive index detector. The mobile phase was 18 mM H₂SO₄ at a flow rate of 6 ml/mm. The concentrations of carbohydrates and organic acids were determined according to a standard calibration curve.

### Example 4A

### Extractive Fermentation-Column Reactor

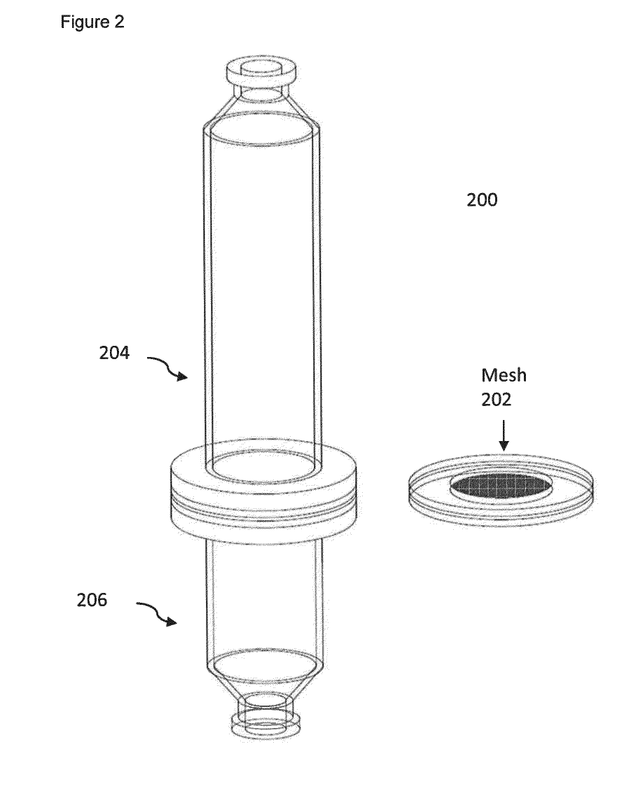

The objective of this experiment was to study the effects of certain extraction variables (oleyl alcohol to trioctylamine ratio, broth to extractant ratio, and extraction time) on extraction efficiency, selectivity of butyric acid, and carbon yields of fermentation. A 100-ml glass column reactor 200 as shown in FIG. 2 was used in this experiment. A piece of round mesh 202 fabricated from stainless steel divided the reactor 200 into two sections: an upper region 204 with a working volume of 70 ml and a lower region 206 with a working volume of 30 ml.

For the extractive fermentation, the lower region 206 of the reactor 200 was inoculated with about 6 g of PVA-immobilized C. cadaveris cell beads prepared as described above and then filled with 60 ml of the basal medium containing 10 g/L glucose as the substrate. The column reactor 200 was then placed still in an anaerobic chamber for a period of time to reduce the concentration of oxygen. Subsequently, 12 ml of the extractant (oleyl alcohol: trioctylamine, 3.24:1) was injected into the upper region 204 of the reactor from an opening located on the top of the column reactor. The reactor 200 was then sealed with an aluminum cap by using a crimper.

In each of the experiment, fermentation was carried out at 37° C. and with stirring (at a speed of 100 rpm in a magnetic stir plate incubator). The extractive fermentation was continued until no detectable amount of butyric acid was further produced due to product inhibition. Each extractive fermentation process was repeated twice for kinetics studies. Samples were taken from the aqueous phase at regular intervals (˜10 hours) for the analysis of free cell density, substrate, and product concentrations in the aqueous phase.

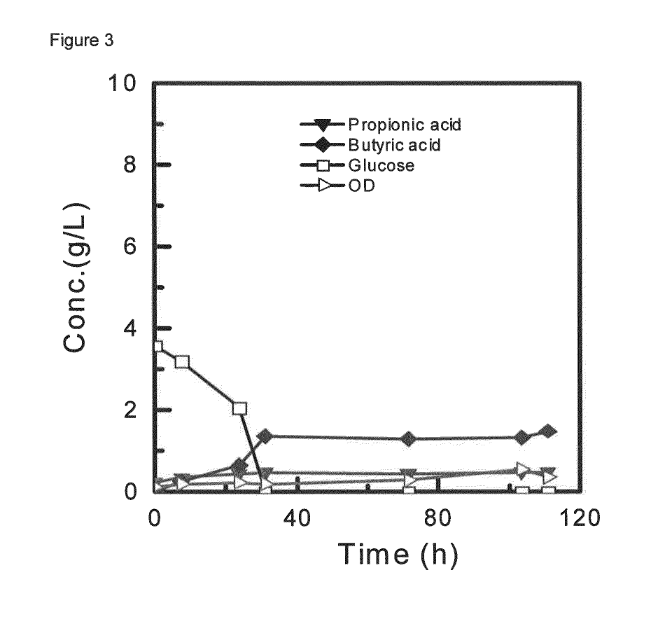

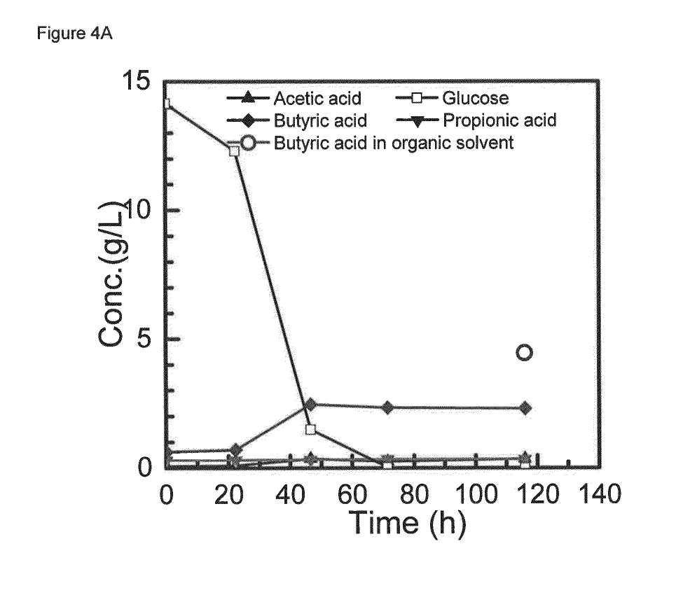

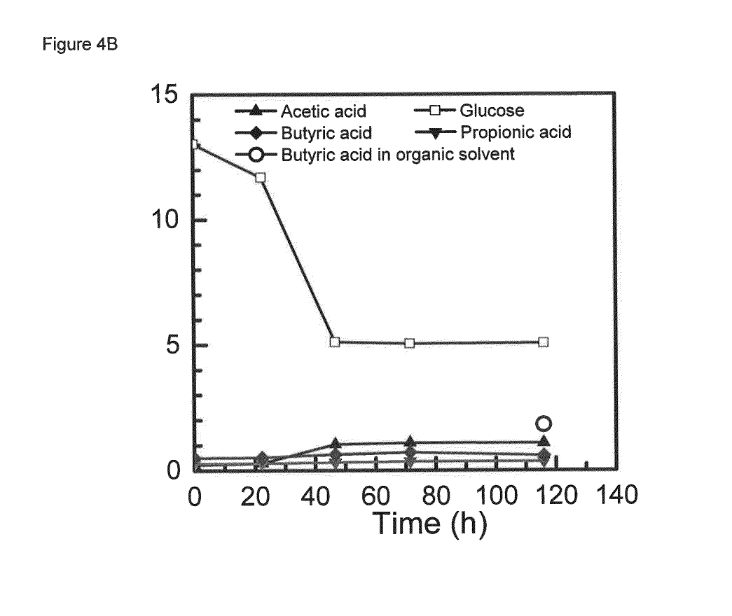

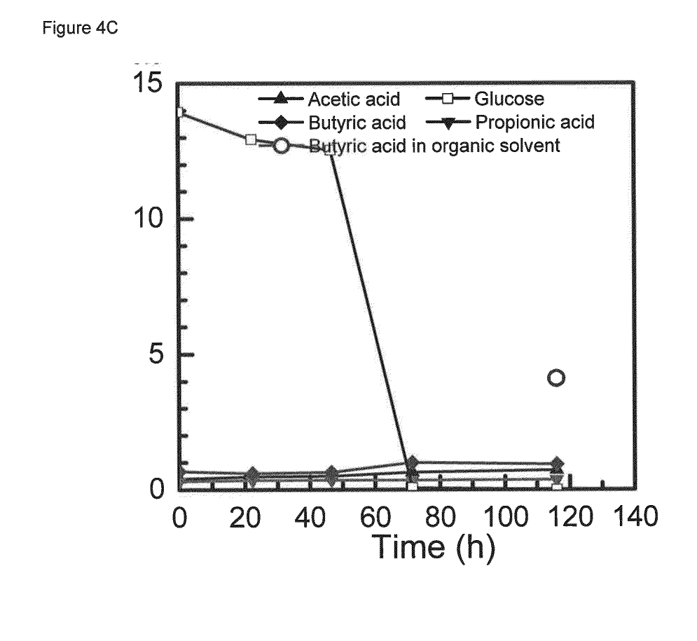

FIG. 3 shows the concentrations of glucose, butyric acid, propionic acid, and OD₆₀₀ in the aqueous phase of the fermentation measured at different time points. The fermentation medium initially contained 3.8 g/L glucose, which was consumed by the C. cadaveris within the first 30 hours of the fermentation. About 110 hours after the fermentation was commenced, the fermentation broth had an optical density (OD₆₀₀) of about 0.56 and about 1.5 g/L of butyric acid.

### Example 4B

An extractive fermentation-column reactor similar to the one shown in FIG. 2 was used in this example. The lower region of the reactor was filled with 50 ml of the basal medium containing 10 g/L glucose and inoculated with about 5 ml of PVA-immobilized clostridium tyrobutyricum. In addition, the upper region of the reactor was filled with 50 ml of 0.7 M trioctylamine in different organic solvents as listed in Table 6. The fermentation was conducted at 37° C. for 100 hours.

As shown in Table 6, no noticeable cell growth was observed after 100 hours when the organic phase of the biphasic culture contained solvents that were commonly used for extraction of butyric acid.

TABLE 6

| | | Methyl | | | | | |
|---|---|---|---|---|---|---|---|
| | | isopropyl | Isoamyl | Ethyl | | Soybean | Cyclohexyl |
| Solvent | N-nonanol | ketone | alcohol | acetate | Toluene | oil | acetate |
| Cells growth | — | — | — | — | — | — | — |

(—) indicated no detectable cell growth.

### Example 4C

An extractive fermentation-column reactor similar to the one shown in FIG. 2 was used in this example, except that the lower region of the reactor was filled with 40 ml of the basal medium containing 10 g/L glucose as the substrate and inoculated with about 4 ml of PVA-immobilized clostridium tyrobutyricum. In addition, the upper region of the reactor was filled with either 40 ml of 1 M TOPO in oleyl alcohol or 40 ml 1 M TBP in oleyl alcohol. The fermentation was conducted at 37° C. for 100 hours.

As the results provided in Table 7 show, the cells were able to grow in the reactor containing 1 M of TOPO in oleyl alcohol as the organic phase but not in 1 M of TBP in oleyl alcohol.

TABLE 7

Extractant

| | Tributylphosphate | Trioctylphosphine Oxide |
|---|---|---|
| solvent | oleyl alcohol | oleyl alcohol |
| Cells growth | 0 | 6.0 |
| (Δ OD₆₀₀) | | |

### Example 4D

An extractive fermentation-column reactor similar to the one shown in FIG. 2 was used in this example. The fermentation medium was prepared by dissolving 12 g of glucose, 4 g of yeast extract, 4 g of peptone, 2.4 g of (NH₄)₂SO₄, 1.2 g of K₂HPO₄, 0.48 g of MgSO₄.7H₂O, and 0.024 g of FeSO₄.7H₂O in 800 ml of distilled water. For the fermentation, 40 ml of the fermentation medium and 4 ml of PVA-immobilized clostridium tyrobutyricum were added to the lower region of the reactor. 40 mL of 1 M of TOPO solution in three different types of solvent (listed in Table 8) was added to the upper region of the reactor. The fermentation was conducted at 37° C. for 40 hours.

The results shown in Table 8 indicate that the cells were able to grow and consumed most of the glucose in the fermentation medium. Additionally, butyric acid had been extracted into the organic phase comprising TOPO.

TABLE 8

| | | | | Conc. of | |
|---|---|---|---|---|---|
| | | | Conc. of | butyric acid | |
| | | | butyric acid in | in the | |
| | Cell | Glucose | the aqueous | organic | Distribution |
| Organic phase | growth | consumption % | phase g/L | phase (g/L) | ratio (D) |
| Oleyl alcohol + TOPO | + | ~100 | 1.6 | 6.3 | 3.86 |
| Soybean oil + TOPO | + | ~100 | 0.8 | 2.6 | 3.25 |
| hexane + TOPO | + | ~100 | 1.1 | 5.2 | 4.58 |

### Example 4E

An extractive fermentation-column reactor similar to the one shown in FIG. 2 was used in this example. The fermentation medium was prepared by dissolving 12 g of glucose, 4 g of yeast extract, 4 g of peptone, 2.4 g of (NH₄)₂SO₄, 1.2 g of K₂HPO₄, 0.48 g of MgSO₄.7H₂O, and 0.024 g of FeSO₄.7H₂O in 800 ml of distilled water. For the fermentation, 40 ml of the fermentation medium and 4 ml of PVA-immobilized clostridium tyrobutyricum were added to the lower region of the reactor. 40 ml of 0.7 M of TOA solution in three different types of solvent (listed in Table 9) was added to the upper region of the reactor. The fermentation was conducted at 37° C. for 40 hours.

The results shown in Table 9 indicate that the cells were able to grow and consumed most of the glucose in the fermentation medium. Additionally, butyric acid had been extracted into the organic phase comprising TOA.

TABLE 9

| | | | Conc. of butyric | Conc. of | |
|---|---|---|---|---|---|
| | | | acid in the | butyric acid in | |
| | Cell | Glucose | aqueous phase | the organic | Distribution |
| Organic phase | growth | consumption % | g/L | phase (g/L) | ratio (D) |
| Oleyl alcohol + TOA | + | ~100 | 1.6 | 5.9 | 3.66 |
| Soybean oil + TOA | + | 30.6 | 1.3 | 0.4 | 0.31 |
| Hexane + TOA | + | 33.4 | 1.9 | 1.2 | 0.65 |

### Example 4F

An extractive fermentation-column reactor similar to the one shown in FIG. 2 was used in this example, except that the lower region of the reactor was filled with 40 ml of the CGM medium containing 15 g/L of glucose as the substrate and inoculated with about 4 ml of PVA-immobilized C. tyrobutyricum ITR104001, C. butyricum ITR104003, or C. tyrobutyricum ITR104004. In addition, the upper region of the reactor was filled with 40 ml of 1 M TOPO in oleyl alcohol. The fermentation was conducted at 37° C. for 115 hours.

FIGS. 4A-C show the concentrations of glucose, acetic acid, butyric acid, propionic acid in the fermentation medium measured at different time points, as well as the concentration of butyric acid in the organic solvent after about 115 hours of fermentation with PVA-immobilized C. tyrobutyricum ITR104001 (FIG. 4A), C. butyricum ITR104003 (FIG. 4B), or C. tyrobutyricum ITR104004 (FIG. 4C).

Table 10 shows the changes of pH in the fermentation medium (i.e., aqueous phase) with different types of bacterias.

TABLE 10

| | C. tyrobutyricum | C. butyricum | C. tyrobutyricum |
|---|---|---|---|
| | ITRI 04001 | ITRI 04003 | ITRI 04004 |
| Initial pH of the | 6.6 | 6.6 | 6.6 |
| fermentation | | | |
| medium (aqueous | | | |
| phase) | | | |
| pH of the | 5.6 | 4.6 | 4.9 |
| fermentation | | | |
| medium at the end | | | |
| of the fermentation | | | |
| Butyric acid | 1.9 | 3.0 | 4.4 |
| distribution | | | |
| factor (D) | | | |

### Example 5

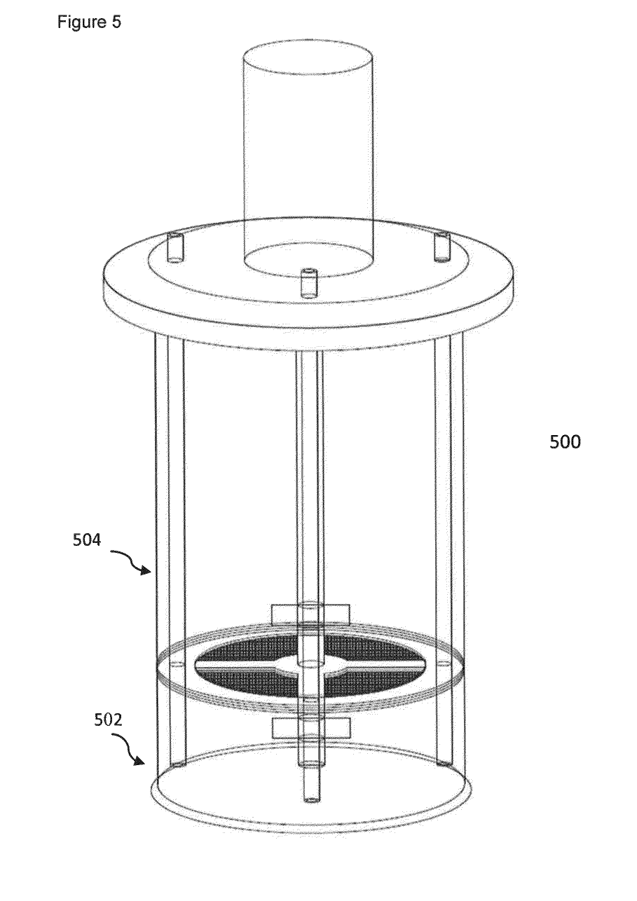

A 2-liter tank fermentor 500 as illustrated in FIG. 5 was used in this Example. A pH meter connecting to an integrated control station (not shown in FIG. 5) was installed within the fermentor 500 for controlling the pH value of the fermentation medium by adding either acid or base solution to the fermentor 500.

For the fermentation, a lower region 502 of the fermentor 500 was filled with about 1.0 liter of the basal medium containing glucose as the substrate. The medium was then purged with N₂ to reach anaerobiosis. The pH of the basal medium was further adjusted to 6.0 with 2 N NaOH. Subsequently, the medium was inoculated with about 100 g of PVA-immobilized C. tyrobutyricum cell beads prepared as described above.

An extractive fermentation-column reactor similar to the one shown in FIG. 5 was used in this example. The lower region of the reactor was filled with 700 ml of the basal medium containing glucose as the substrate and inoculated with about 70 ml of PVA-immobilized clostridium tyrobutyricum. In addition, the upper region of the reactor was filled with either 700 ml of 1 M TOPO in oleyl alcohol or 700 ml of oleyl alcohol without TOPO. The fermentation was conducted at 37° C. for 40 hours, and the pH of the fermentation medium was not controlled in this example.

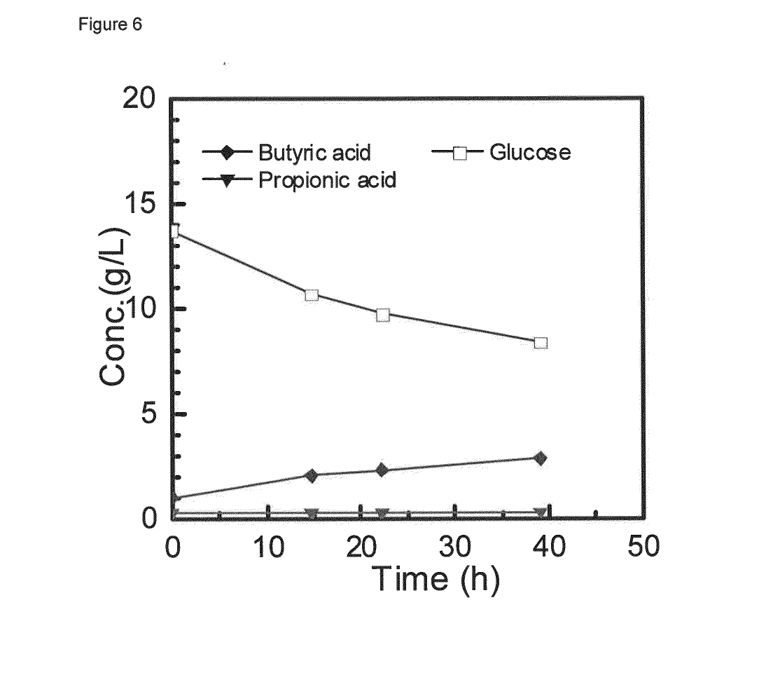

FIG. 6 shows the concentrations of glucose, butyric acid, and propionic in the basal medium at various time points and in the reactor that contained only oleyl alcohol as the organic phase. The pH of the basal medium was 6.0 at the beginning of the fermentation and dropped to 4.2 at the end of the fermentation.

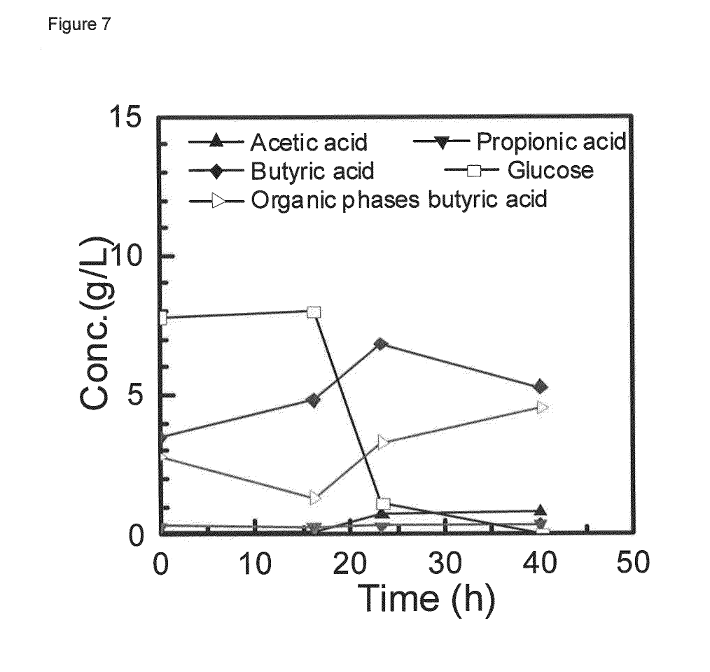

FIG. 7 shows the concentrations of glucose, butyric acid, and propionic in the basal medium at various time points and in the reactor that contained 1 M TOPO in oleyl alcohol as the organic phase. The pH of the basal medium was 6.0 at the beginning of the fermentation and dropped to about 5.0 at the end of the fermentation.

Table 11 below provides a side-by-side comparison of the fermentation results with different organic phases at the upper region of the reactor.

TABLE 11

| | Organic Phase | |
|---|---|---|
| | 1M TOPO in | |
| | oleyl alcohol | Oleyl alcohol only |
| pH | Dropped from | Dropped from about |
| | about 6.0 to | 6.0 to about 5.0 |
| | about 5.0 | |
| Total volume of the reactor | 2000 mL | 2000 mL |
| % of glucose consumed | 38.68 | 100 |
| Distribution ratio of butryic acid | — | 0.86 |
| Butryic acid concentration in | NA/2.9 | 4.55/5.27 |
| the organic phase/Butryic acid | | |
| concentration in the aqueous | | |
| phase (g/L) | | |

It will be apparent to those skilled in the art that various modifications and variations can be made to the disclosed embodiments. It is intended that the specification and examples be considered as exemplary only, which do not limit the scope of the disclosure.

What is claimed is:

1. A fermentation process for making carboxylic acids and/or alcohols, comprising:
growing an microorganism capable of producing carboxylic acids and/or alcohols in an aqueous medium and in the presence of an organic medium, and
recovering the carboxylic acids and/or alcohols from the organic medium;
wherein a mesh is placed at an interface of the organic medium and the aqueous medium; and
further wherein the organic medium comprises at least one organic solvent and at least one extractant chosen from tri-alkylphosphine oxides and tri-alkylamines.

2. The fermentation process according to claim 1, wherein the microorganism is chosen from C. tyrobutyricum, C. thermobutyricum, C. butyricum, C. populeti, C. cadaveros, C. cellobioparum, C. cochlearium, C. pasteurianum, C. roseum, C. rubrum, C. sporogenes, C. acetobutyricum, C. beijerinckii, Clostridium aurantibutyricum, and C. tetanomorphum.

3. The fermentation process according to claim 1, wherein the at least one organic solvent is chosen from C₈-C₁₈ alcohols, triglyceride, C₆-C₁₆ alkanes and fatty acid methyl esters.

4. The fermentation process according to claim 1, wherein the at least one organic solvent is chosen from oleyl alcohol, hexane, and soybean oil.

5. The fermentation process according to claim 1, wherein the at least one extractant is chosen from trioctylphosphine oxide and trioctylamine.

6. The fermentation process according to claim 5, wherein the at least one extractant is trioctylphosphine oxide.

7. The fermentation process according to claim 6, wherein the trioctylphosphine oxide is present in the organic medium in a concentration of less than or equal to 1 M.

8. The fermentation process according to claim 5, wherein the at least one extractant is trioctylamine.

9. The fermentation process according to claim 8, wherein the trioctylamine is present in the organic medium in a concentration of less than or equal to 0.7 M.

10. The fermentation process according to claim 1, wherein the pH of the aqueous medium is in a range of about 3 to about 6.

11. The fermentation process according to claim 1, wherein the carboxylic acids comprise at least one acid chosen from acetic, propionic, butyric, fumaric, malic, acrylic, citric, gluconic, and itaconic acid.

12. The fermentation process according to claim 1, wherein the alcohols comprise at least one alcohol chosen from ethanol, propanol, and butanol.

13. A fermentation process for making butyric acid, comprising:
growing an microorganism capable of producing butyric acid in an aqueous medium and in the presence of an organic medium, and recovering the butyric acid from the organic medium;
wherein a mesh is placed at an interface of the organic medium and the aqueous medium; and
further wherein the organic medium comprises at least one organic solvent and at least one extractant chosen from tri-alkylphosphine oxides and tri-alkylamines.

14. The fermentation process according to claim 13, wherein the at least one organic solvent is chosen from oleyl alcohol, hexane, and soybean oil.

15. The fermentation process according to claim 13, wherein the at least one extractant is chosen from trioctylphosphine oxide and trioctylamine.

16. The fermentation process according to claim 15, wherein the at least one extractant is trioctylphosphine oxide.

17. The fermentation process according to claim 16, wherein the trioctylphosphine oxide is present in the organic medium in a concentration of less than or equal to 1 M.

18. The fermentation process according to claim 15, wherein the at least one extractant is trioctylamine.

19. The fermentation process according to claim 18, wherein the trioctylamine is present in the organic medium in a concentration of less than or equal to 0.7 M.
